(19) United States
(12) Patent Application Publication
(10) Pub. No.: US 2024/0033573 A1
(43) Pub. Date: Feb. 1, 2024

(54) Electronic Devices With Improved Aerobic Capacity Estimation

(71) Applicant: Apple Inc., Cupertino, CA (US)

(72) Inventors: Aditya Sarathy, San Jose, CA (US); Bharath Narasimha Rao, San Mateo, CA (US); Umamahesh Srinivas, San Jose, CA (US)

(21) Appl. No.: 18/298,183

(22) Filed: Apr. 10, 2023

Related U.S. Application Data
(60) Provisional application No. 63/392,786, filed on Jul. 27, 2022.

(51) Int. Cl. A63B 24/00; A63B 22/02

(52) U.S. Cl. CPC A63B 24/0062; A63B 22/02; A63B 2230/06; A63B 2220/30; A63B 2220/16; A63B 2220/833; A63B 2220/803; A63B 2024/0068

(57) ABSTRACT

An electronic device may use motion and/or activity sensors to estimate a user's maximum volumetric flow of oxygen (VO₂ max). In particular, the electronic device may use the user's heart rate, speed, and grade to determine the VO₂ max. However, in indoor environments, it may be difficult to accurately measure the user's speed and grade. Therefore, the device may receive the speed and grade from external equipment, such as exercise equipment. To ensure that the user is moving at the reported speed, a discordance detector may compare the user's cadence to an expected cadence based on the speed and grade reported by the external equipment. If the user's cadence is within an acceptable range of the expected cadence, the user's VO₂ max may be estimated based on the speed and grade. If the user's cadence is not within the acceptable range, the speed and grade may be discarded or corrected.

This application claims the benefit of provisional application No. 63/392,786, filed Jul. 27, 2022, which is hereby incorporated by reference herein in its entirety.

### FIELD

This relates generally to electronic devices, and, more particularly, to electronic devices with health sensor and detection circuitry.

### BACKGROUND

Electronic devices may be used to track a user's health and activity, such as exercise activity, using sensors, such as heart rate and motion sensors. Information produced by these sensors may be used to estimate the user's maximum volumetric flow of oxygen (VO₂ max). However, it may be difficult to measure the user's motion accurately in some situations, such as when the user is exercising indoors.

### SUMMARY

Electronic devices such as cellular telephone, wristwatches, and other portable devices are often worn or carried by users. The electronic devices may include motion sensors, such as accelerometers, gyroscopes, and/or global positioning system (GPS) sensors, as examples, that may indicate movement of the electronic device. Additionally, the devices may include health sensors, such as heart rate sensors, electrocardiogram sensors, and/or perspiration sensors, as examples, that may indicate activity information of the user.

To estimate a user's maximum volumetric flow of oxygen, or VO₂ max, control circuitry within the electronic devices may rely on both the movement of the electronic device and the activity information of the user. In particular, the VO₂ max may be determined based on the user's speed and grade (e.g., the slope on which they are moving), as well as their heart rate. However, in some situations, it may be difficult to measure the user's speed and grade accurately. In particular, if the user is exercising indoors, such as on exercise equipment, it may be difficult for the motion sensors to determine the speed and grade. Therefore, the speed and grade information may be obtained from external equipment, such as the exercise equipment or a camera system.

To ensure that the received speed and grade information is accurate (e.g., that the user is moving at the same speed and grade as indicated by the external equipment), the user's cadence may be measured by motion sensors in the electronic device and compared to an expected cadence range based on the received speed and grade information. If the user's cadence is within the expected cadence range, the received speed and grade information may be used to estimate the user's VO₂ max. If the user's cadence is not within the expected cadence range, the received speed and grade information may be discarded or corrected to ensure an accurate VO₂ max estimate.

### BRIEF DESCRIPTION OF THE DRAWINGS

FIG. 1 is a drawing of an illustrative wearable electronic device in accordance with an embodiment.

FIG. 2 is a diagram of an illustrative system of an electronic device in communication with external equipment in accordance with an embodiment.

FIG. 3 is a diagram of an illustrative motion sensor apparatus and associated circuitry in accordance with an embodiment.

FIG. 4 is a diagram of an illustrative activity sensor apparatus and associated circuitry in accordance with an embodiment.

FIG. 5 is a diagram of illustrative components used by control circuitry to estimate a user's VO₂ max in accordance with an embodiment.

FIG. 6 is a flowchart of illustrative steps that may be used to calculate a user's VO₂ max in varying conditions.

FIG. 7 is a graph of an illustrative relationship between a user's cadence and the speed of a fitness machine in accordance with an embodiment.

FIG. 8 is a graph of an illustrative arm angle of a user over time in accordance with an embodiment.

### DETAILED DESCRIPTION

Electronic devices are often carried by users as they conduct their daily activities. For example, a user may carry an electronic device while walking, exercising, or climbing stairs. To provide a user with fitness tracking functionality and other functions, it may be desirable to monitor a user's activities. For example, sensors in an electronic device may monitor user movement and level of exertion. In an illustrative configuration, a motion sensor such as an accelerometer, a gyroscope, an altimeter, and/or other sensors in an electronic device may be used in determining when a user has climbed a flight of stairs or performed other physical activities, and a health sensor such as a heart rate sensor may measure a user's heart rate. The same sensors and/or other sensors within the device may be used to determine whether a user has been active or exercised, and the device may track the user's workouts.

In some cases, it may be desirable to calculate a VO₂ max value for a user. While a VO₂ max value may be calculated based on a user's workout data obtained by the electronic device, some users may desire to exercise indoors, such as on a treadmill or other indoor workout equipment. In indoor environments, it may be difficult to determine the speed with which the user is moving on the workout equipment and other characteristics of the equipment, such as the grade that the user is running up. In these situations, it may be desirable to incorporate data from the workout equipment. For example, the electronic device may receive information from the workout equipment, such as speed and grade information. The speed and grade information may be combined with heart rate, cadence, and/or other information generated by the electronic device to ensure that the user is exerting an expected amount of effort given the information from the workout equipment (e.g., that the user is running at an expected cadence or speed based on the speed and grade of a treadmill). The electronic device may then use the speed and grade information, the heart rate, the cadence, and/or other desired information to calculate a VO₂ max value.

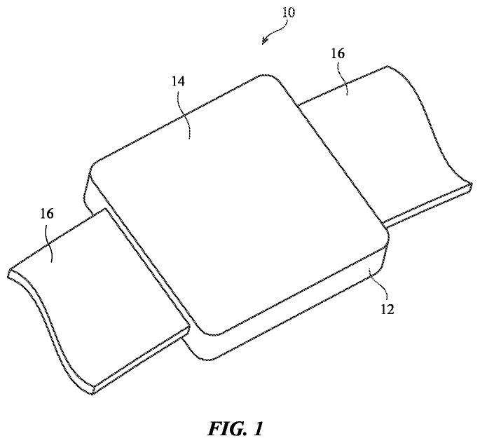

In general, any suitable electronic devices may be used in measuring the user's motion and activity. As shown in FIG. 1, a wearable electronic device 10, which may be a wristwatch device, may have a housing 12, a display 14, and a strap 16. The wristwatch may attach to a user's wrist via strap 16, and provide skin contact on the user's wrist, by which sensors within device 10 may measure signs of physical assertion, such as increased heart rate and cadence. Additionally, sensors within housing 12 may be used to determine that the wristwatch, and therefore the user, is moving.

Display 14 may be a display of any desired display technology, such as an organic light-emitting diode display, a microLED display, or other displays with light-emitting diodes, a liquid crystal display, or other display. Displays 14 may be touch sensitive (e.g., display 14 may include two-dimensional touch sensors for capturing touch input from a user) and/or display 14 may be insensitive to touch.

Housing 12 may be formed from any desired material, such as metal, plastic, or other material. Other components, such as display driver circuitry, communications circuitry, control circuitry, health sensor circuitry, environmental sensor circuitry, and other circuitry may be formed within housing 12, if desired.

Although electronic device 10 is shown as being a wearable electronic device, this is merely illustrative. In general, electronic device 10 may be any desired device. In some embodiments, electronic device 10 may be a cellular telephone, which may be carried within a user's pocket and gather data when the user moves.

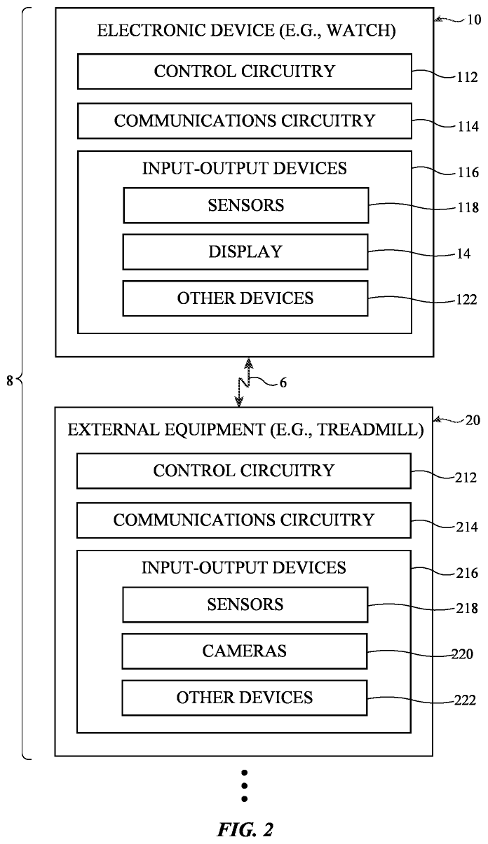

Regardless of the type of device of electronic device 10, it may be desirable to determine health information, such as a user's VO₂ max value, using sensors within electronic device 10. In some circumstances, such as when a user is exercising indoors, it may be desirable to receive additional information from external equipment to determine health information more accurately. A diagram of electronic device 10 in communication with external equipment, such as exercise equipment, is shown in FIG. 2.

As shown in FIG. 2, electronic device 10 and external equipment 20, as well as additional electronic devices and/or external equipment, may be used in system 8, if desired. Device 10 may be, for example, a wristwatch device as shown in FIG. 1, or may be a cellular telephone, a media player, or other handheld or portable electronic device, a wristband device, a pendant device, a headphone, ear bud, or earpiece device, a head-mounted device such as glasses, goggles, a helmet, or other equipment worn on a user's head, or other wearable or miniature device, a navigation device, or other accessory, and/or equipment that implements the functionality of two or more of these devices. Illustrative configurations in which electronic device 10 is a portable electronic device such as a cellular telephone, wristwatch, or portable computer may sometimes be described herein as an example.

Electronic devices in system 8, such as electronic device 10, may have control circuitry 112. Control circuitry 112 may include storage and processing circuitry for controlling the operation of device 10. Circuitry 112 may include storage such as hard disk drive storage, nonvolatile memory (e.g., electrically-programmable-read-only memory configured to form a solid-state drive), volatile memory (e.g., static or dynamic random-access-memory), etc. Processing circuitry in control circuitry 112 may be based on one or more microprocessors, microcontrollers, digital signal processors, baseband processors, power management units, audio chips, graphics processing units, application specific integrated circuits, and other integrated circuits. Software code may be stored on storage in circuitry 112 and run on processing circuitry in circuitry 112 to implement control operations for device 10 (e.g., data gathering operations, operations involving the adjustment of the components of device 10 using control signals, etc.).

Electronic device 10 may include wired and wireless communications circuitry. For example, electronic device 10 may include radio-frequency transceiver circuitry such as cellular telephone transceiver circuitry, wireless local area network transceiver circuitry (e.g., WiFi® circuitry), short-range radio-frequency transceiver circuitry that communicates over short distances using ultra high frequency radio waves (e.g., Bluetooth® circuitry operating at 2.4 GHz or other short-range transceiver circuitry), millimeter wave transceiver circuitry, and/or other wireless communications circuitry.

Device 10 may include input-output devices 116. Input-output devices 116 may be used to allow a user to provide device 10 with user input. Input-output devices 116 may also be used to gather information on the environment in which device 10 is operating. Output components of input-output devices 116 may allow device 10 to provide a user with output and may be used to communicate with external electrical equipment.

As shown in FIG. 2, input-output devices 116 may include one or more optional displays such as display 14. Display 14 may be an organic light-emitting diode display, a microLED display, or other display with light-emitting diodes, a liquid crystal display, or other display. Display 14 may be touch sensitive (e.g., display 14 may include two-dimensional touch sensors for capturing touch input from a user) and/or display 14 may be insensitive to touch.

Input-output devices 116 may include sensors 118. Sensors 118 may include, for example, three-dimensional sensors (e.g., three-dimensional image sensors such as structured light sensors that emit beams of light and that use two-dimensional digital image sensors to gather image data for three-dimensional images from light spots that are produced when a target is illuminated by the beams of light, binocular three-dimensional image sensors that gather three-dimensional images using two or more cameras in a binocular imaging arrangement, three-dimensional lidar (light detection and ranging) sensors, three-dimensional radio-frequency sensors, or other sensors that gather three-dimensional image data), cameras (e.g., infrared and/or visible digital image sensors), gaze tracking sensors (e.g., a gaze tracking system based on an image sensor and, if desired, a light source that emits one or more beams of light that are tracked using the image sensor after reflecting from a user's eyes), touch sensors, capacitive proximity sensors, light-based (optical) proximity sensors, other proximity sensors, force sensors, sensors such as contact sensors based on switches, gas sensors, pressure sensors, moisture sensors, magnetic sensors (e.g., a magnetometer), audio sensors (microphones), ambient light sensors, microphones for gathering voice commands and other audio input, sensors that are configured to gather information on motion, position, and/or orientation (e.g., accelerometers, gyroscopes, pressure sensors, compasses, and/or inertial measurement units that include all of these sensors or a subset of one or two of these sensors), health sensors that measure various biometric information (e.g., heartrate sensors, such as a photoplethysmography sensor), electrocardiogram sensors, and perspiration sensors) and/or other sensors.

User input and other information may be gathered using sensors and other input devices in input-output devices 116. For example, sensors 118 may include health sensors, such as heart rate sensors, and motion sensors, such as accelerometers and/or gyroscopes. In some embodiments, sensors 118 may detect a user's heart rate and cadence using these sensors.

If desired, input-output devices 116 may include other devices 122 such as haptic output devices (e.g., vibrating components), light-emitting diodes and other light sources, speakers such as ear speakers for producing audio output, circuits for receiving wireless power, circuits for transmitting power wirelessly to other devices, batteries and other energy storage devices (e.g., capacitors), joysticks, buttons, and/or other components.

As indicated by arrow 6, electronic device 10 may be in communication with external equipment 20. External equipment 20 may be, for example, workout/exercise equipment, camera equipment, or other desired equipment. In some illustrative examples, external equipment 20 may be a treadmill.

External equipment 20 may have control circuitry 212. Control circuitry 212 may include storage and processing circuitry for controlling the operation of equipment 20. Circuitry 112 may include storage such as hard disk drive storage, nonvolatile memory (e.g., electrically-programmable-read-only memory configured to form a solid-state drive), volatile memory (e.g., static or dynamic random-access-memory), etc. Processing circuitry in control circuitry 212 may be based on one or more microprocessors, microcontrollers, digital signal processors, baseband processors, power management units, audio chips, graphics processing units, application specific integrated circuits, and other integrated circuits. Software code may be stored on storage in circuitry 212 and run on processing circuitry in circuitry 212 to implement control operations for equipment 20 (e.g., data gathering operations, operations involving the adjustment of the components of equipment 20 using control signals, etc.). In some embodiments, external equipment 20 may be a treadmill, and control circuitry 212 may control a speed, grade, and/or other settings of the treadmill. The speed and grade information may be stored within storage circuitry of control circuitry 212.

Equipment 20 may include wired and wireless communications circuitry. For example, equipment 20 may include radio-frequency transceiver circuitry such as cellular telephone transceiver circuitry, wireless local area network transceiver circuitry (e.g., WiFi® circuitry), short-range radio-frequency transceiver circuitry that communicates over short distances using ultra high frequency radio waves (e.g., Bluetooth® circuitry operating at 2.4 GHz or other short-range transceiver circuitry), millimeter wave transceiver circuitry, and/or other wireless communications circuitry.

Input-output devices 216 may include sensors 218. Sensors 218 may include, for example, three-dimensional sensors (e.g., three-dimensional image sensors such as structured light sensors that emit beams of light and that use two-dimensional digital image sensors to gather image data for three-dimensional images from light spots that are produced when a target is illuminated by the beams of light, binocular three-dimensional image sensors that gather three-dimensional images using two or more cameras in a binocular imaging arrangement, three-dimensional lidar (light detection and ranging) sensors, three-dimensional radio-frequency sensors, or other sensors that gather three-dimensional image data), cameras (e.g., infrared and/or visible digital image sensors), gaze tracking sensors (e.g., a gaze tracking system based on an image sensor and, if desired, a light source that emits one or more beams of light that are tracked using the image sensor after reflecting from a user's eyes), touch sensors, capacitive proximity sensors, light-based (optical) proximity sensors, other proximity sensors, force sensors, sensors such as contact sensors based on switches, gas sensors, pressure sensors, moisture sensors, magnetic sensors (e.g., a magnetometer), audio sensors (microphones), ambient light sensors, microphones for gathering voice commands and other audio input, sensors that are configured to gather information on motion, position, and/or orientation (e.g., accelerometers, gyroscopes, pressure sensors, compasses, and/or inertial measurement units that include all of these sensors or a subset of one or two of these sensors), health sensors that measure various biometric information (e.g., heartrate sensors, such as a photoplethysmography sensor), electrocardiogram sensors, and perspiration sensors) and/or other sensors.

Input-output devices 216 may include cameras 220. For example, a treadmill or other workout equipment may include at least one camera, or external equipment 20 may be a camera system. In some embodiments, cameras 220 may be used to determine the speed and grade information of a user exercising indoors on gym equipment.

User input and other information may be gathered using sensors and other input devices in input-output devices 216. If desired, input-output devices 216 may include other devices 222. such as haptic output devices (e.g., vibrating components), light-emitting diodes and other light sources, speakers such as ear speakers for producing audio output, circuits for receiving wireless power, circuits for transmitting power wirelessly to other devices, batteries and other energy storage devices (e.g., capacitors), joysticks, buttons, and/or other components.

During operation, the communications circuitry of the devices and external equipment in system 8 (e.g., communications circuitry 112 and communications circuitry 212), may be used to support communication between the electronic devices. Communication between device 10 and external equipment 20 is shown by arrow 6 of FIG. 2.

In some embodiments, communications circuitry 212 may be used to send information to device 10 (i.e., communications circuitry 112 of device 10). For example, if a user is using workout equipment, such as a treadmill, it may be desirable for device 10 to obtain information regarding the speed and grade of the workout equipment while the user is exercising. Therefore, external equipment 20 may send speed and grade information to device 10. The speed and grade information may then be used to determine the user's VO₂ max.

In general, the speed and grade information (or other information) may be communicated from equipment 20 to device 10 in any desired manner. For example, Bluetooth® circuitry, near-field communications (NFC) circuitry, or other circuitry may be used to communicate information between equipment 20 and device 10. Electronic devices in system 8 may use wired and/or wireless communications circuitry to communicate through one or more communications networks (e.g., the Internet, local area networks, etc.). The communications circuitry may be used to allow data to be transmitted to and/or received by device 10 from external equipment 20 and/or to provide data to external equipment 20.

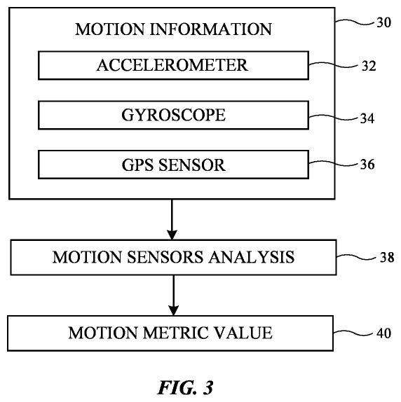

In some embodiments, device 10 (and/or other devices within system 8) may determine a motion of a user. As shown in FIG. 3, motion information 30 may be determined using one or more sensors, such as sensors 118 of device 10. Sensors 118 may include one or more of accelerometer 32, gyroscope 34, and global positioning system (GPS) sensor 36 to measure motion information 30, as examples. Accelerometer 32 may be a two-dimensional or three-dimensional accelerometer (e.g., accelerometer 32 may measure motion in two directions or three directions). In some embodiments, sensors 118 may include other motion sensors or other sensors that may be used to detect motion more generally, such as pressure sensors, altimeters, orientation sensors, cameras, light sensors, microphones, or other sensors. However, this is merely illustrative. In general, sensors 118 may include any desired sensors to measure motion of the associated device.

Using data generated by the sensors that collect the motion information, control circuitry, such as control circuitry 112 of device 10, may perform a motion sensor analysis 38 by analyzing the data generated by the one or more sensors. For example, the control circuitry may compare the data generated by each sensor and fuse the data to determine a motion metric value 40. This may be done statistically through weighting, removing outlier measurements from the set, averaging the data, or any other desired method. Motion metric value 40 may be stored within the storage circuitry of the electronic device.

In general, the sensors used to calculate motion metric value 40 may automatically obtain updated motion data at any desired time interval and/or be manually triggered by actions of a user. In either case, the motion metric value 40 may be updated and logged within the storage circuitry when there is enough data to calculate the metric value.

In some embodiments, motion metric value 40 may include information on a user's cadence. For example, accelerometer 32, gyroscope 34, and/or other sensors 118 may be used to determine a user's cadence. The user's cadence may be the user's steps-per-minute, as an example. Alternatively or additionally, motion metric value 40 may include information on a user's arm angle. For example, accelerometer 32, an orientation sensor, and/or other sensors 118 may be used to determine a user's arm angle.

Motion metric value 40 may also include information regarding the speed with which the user is moving (e.g., based on a speed of device 10 as it is being carried by the user) and/or the grade up which the user is moving (e.g., based on a ratio of the vertical and horizontal displacement of device 10 as it is being carried by the user). However, these metrics are merely illustrative. In general, any desired motion metric values 40 may be generated using sensors 118.

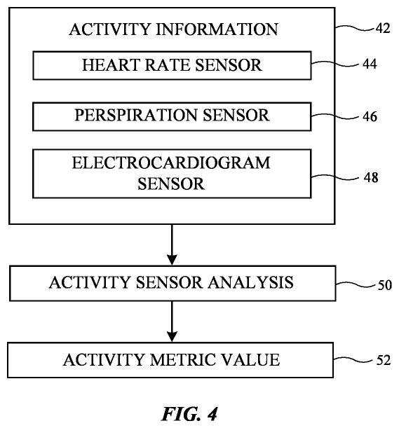

In addition to calculating the motion of the device, sensors with electronic device 10 may determine activity information of the user. As shown in FIG. 4, activity information 42 may be determined by one or more sensors, such as sensors 118 of device 10. Sensors 118 may include one or more of heart rate sensor 44, perspiration sensor 46, and electrocardiogram (ECG) sensor 48 to measure activity information 42, as examples. These sensors may also be used in conjunction with the motion sensors described in connection with FIG. 3, if desired.

Using data generated by the activity information sensors (and the motion information sensors, if desired), control circuitry, such as control circuitry 112 of device 10, may perform an activity sensor analysis 50 by analyzing the data generated by the one or more sensors. For example, the control circuitry may compare the data generated by each sensor and fuse the data to determine an activity metric value 52. This may be done statistically through weighting, removing outlier measurements from the set, averaging the data, or any other desired method. Activity metric value 52 may be stored within the storage circuitry of the electronic device.

In general, the sensors used to calculate activity metric value 52 may automatically obtain updated motion data at any desired time interval and/or be manually triggered by actions of a user. In one example, the electronic device may be placed into an exercise mode, in which the activity information sensors and/or the motion sensors are activated more frequently to determine the user's biometric information more often. In any case, the activity metric value 52 may be updated and logged within the storage circuitry when there is enough data to calculate the metric value.

Activity metric value 52 may include a user's heart rate, perspiration, blood oxygen, and/or any other desired metric obtained using sensors 118.

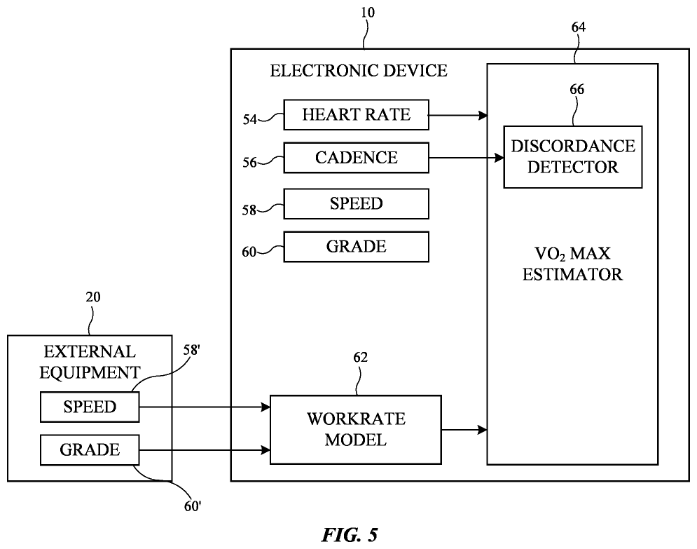

Based on motion metric value 40 and activity metric value 52, a user's VO₂ max value may be calculated or estimated. However, if a user is exercising indoors or otherwise exercising on exercise equipment, such as on a treadmill, it may be difficult for sensors within device 10 to determine some of the motion data. In particular, it may be difficult for sensors within device 10 to measure the speed with which a user moves on exercise equipment and the grade settings of the exercise equipment. Therefore, it may be desirable to obtain speed and grade information from external equipment, such as from the exercise equipment itself. An example of calculating a VO₂ max value based on information from the electronic device and from external equipment is shown in FIG. 5.

As shown in FIG. 5, electronic device 10 may generate heart rate 54, cadence 56 (also referred to as cadence data herein), speed 58, and grade 60. Although all of these metrics may be used to determine a user's VO₂ max, it may be desirable to obtain some of the information, such as speed and grade information from external equipment. Therefore, external equipment 20 may generate speed 58′ and grade 60′ (also referred to as speed and grade information herein). In some embodiments, external equipment 20 may be exercise equipment, such as a treadmill, on which the user sets the speed and grade for their workout. In that case, circuitry within external equipment 20 may store the speed 58′ and grade 60′ after they are set by the user, and communicate speed 58′ and grade 60′ to electronic device 10. Alternatively or additionally, external equipment 20 may include cameras or other sensors that monitor a user and exercise equipment. In that case, the cameras or other sensors may determine speed 58′ and grade 60′ by taking videos, images, or otherwise monitoring the user and processing the image data or other data to determine the speed and grade.

Regardless of how speed 58′ and grade 60′ are obtained, speed 58′ and grade 60′ may be communicated to electronic device 10, such as over bidirectional link 6 of FIG. 2. Once speed 58′ and grade 60′ have been received by electronic device 10, speed 58′ and grade 60′ may be used to determine a workrate of the user using workrate model 62. Workrate model 62 may be used to calculate the user's workrate (e.g., the work done by user while exercising) using speed 58′ and grade 60′, along with characteristics of the user, such as their height, weight, age, and/or any other characteristics, if desired.

Heart rate 54 and the output of workrate model 62 may then be used to estimate the user's VO₂ max in VO₂ max estimator 64. VO₂ max estimator 64 may be a part of control circuitry 112, if desired. In operation, the heart rate of the user needed to output the given workrate may be used to estimate the user's VO₂ max. However, in some circumstances, the output of workrate model 62 may be inaccurate. Because workrate model 62 is dependent upon speed 58′ and grade 60′ from external equipment 20, it may not reflect the actual speed of the user. For example, a user may step off of external equipment 20 or hold onto handles or other supports on external equipment 20. In this way, external equipment 20 may maintain a speed (such as a belt speed on a treadmill), while a user is not moving at the same speed. Therefore, it may be desirable to ensure that the user is active at the same pace reported by the external equipment.

As shown in FIG. 5, discordance detector 66 may be used to ensure that the estimated VO₂ max is accurate (e.g., that the user is moving on the exercise equipment at the same speed as reported by the equipment). Discordance detector 66 may be integrated with VO₂ max estimator 64, as shown in FIG. 5, or may be implemented separately from VO₂ max estimator 64. In operation, discordance detector 66 may analyze cadence 56 (which may be a part of motion metric value 40) to see if it is within an expected range (i.e., within a range of an expected cadence). For example, discordance detector 66 may calculate an expected cadence range based on speed 58′ and grade 60′ (and other characteristics, such as characteristics of the user, if desired). If cadence 56 is within the expected cadence range, then VO₂ max estimator 64 may keep the estimate of the user's VO₂ max. Alternatively, if cadence 56 is outside of the expected cadence range, then VO₂ max estimator 64 may disregard the estimate of the user's VO₂ max or apply a corrective factor to the user's VO₂ max estimate. In this way, the user's VO₂ max may be estimated based on their exercise on indoor exercise equipment.

Although not shown in FIG. 5, VO₂ max estimator 64 may also utilize previous VO₂ max estimations for a user in calculating an updated VO₂ max estimation.

Although workrate model 62 and VO₂ max estimator 64 are both shown as being within electronic device 10, this is merely illustrative. In general, the workrate and/or VO₂ max may be calculated on additional equipment and/or on electronic device 10, if desired.

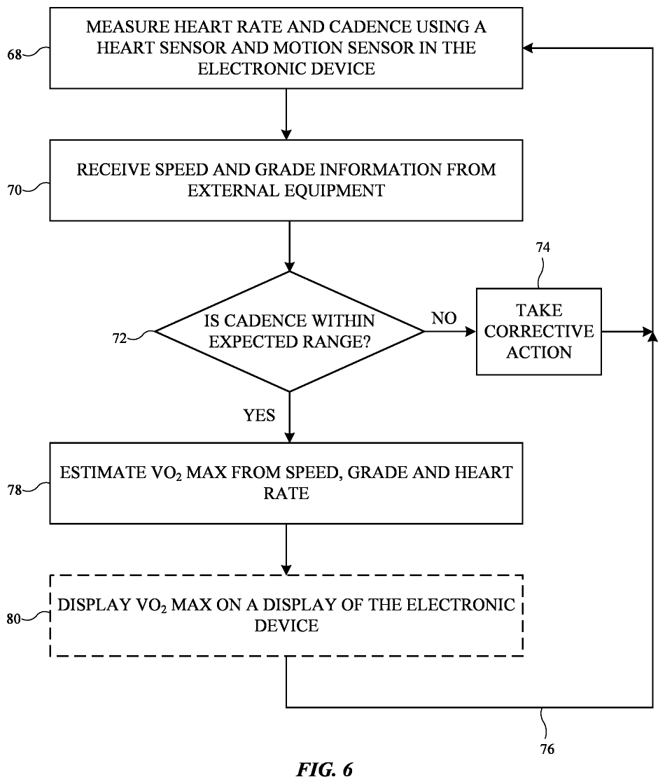

Additionally, although FIG. 5 shows heart rate 54 gathered by electronic device 10, heart rate 54 may be gathered fully or partially by another device, if desired. For example, a user's heart rate may be gathered using a chest strap, electrodes on workout equipment (such as on external equipment 20), or other desired equipment. In some embodiments, the heart rate may be gathered using a chest strap, while the speed and grade information are received from external equipment 20, which may be exercise equipment, such as a treadmill, or may be a camera system that monitors a user. However, this is merely illustrative. In general, a user's heart rate, such as heart rate 54, may be gathered in any desired manner. Regardless of which equipment or devices are used to gather a user's hear rate, cadence, speed, and grade, a method of calculating a VO₂ max is shown in FIG. 6.

As shown in FIG. 6, at step 68, a user's heart rate and cadence may be measured. For example, a heart rate sensor (such as heart rate sensor 44) and motion sensors (such as accelerometer 32) in a wearable electronic device, such as a watch, may be used to determine the heart rate and cadence, respectively.

At step 70, either after step 68 or concurrently with at least a portion of step 68, the electronic device may receive speed and grade information (such as speed 58′ and grade 60′) from external equipment (such as external equipment 20). The speed and grade information may be received using communications circuitry, such as communications circuitry 114.

At step 72, it may be determined whether the cadence is within an expected range. In particular, a discordance detector (such as discordance detector 66) in the electronic device or on an external device may be used for this determination. The discordance detector may determine the expected cadence range from the received speed and grade information (and characteristics of the user, such as height, weight, and/or age information, if desired).

If the cadence is not within the expected range, at step 74, corrective action may be taken. For example, the speed and grade information may be discarded (i.e., a VO₂ max value may not be estimated with the speed and grade information during time periods in which the cadence is not within an expected range). Alternatively, the speed and grade information may be adjusted based on the user's cadence and/or other data generated by the electronic device. For example, if the user's cadence is below the expected cadence range by 10%, the speed and/or grade information may be corrected by a corresponding amount, such as by 10%. However, this relationship is merely illustrative. In general, any desired method of correcting or discarding the speed and grade information may be used in response to determining that the cadence is outside of the expected range.

If the cadence is within the expected range (or if the speed and grade information is corrected, rather than discarded), at step 78, the user's VO₂ max may be estimated based on the speed, grade, and heart rate information. In particular, a workrate model, such as workrate model 62, may be used to determine the work expended by the user while exercising, and a VO₂ max estimator, such as VO₂ max estimator 64, may be used to estimate the user's VO₂ max based on the workrate and heart rate.

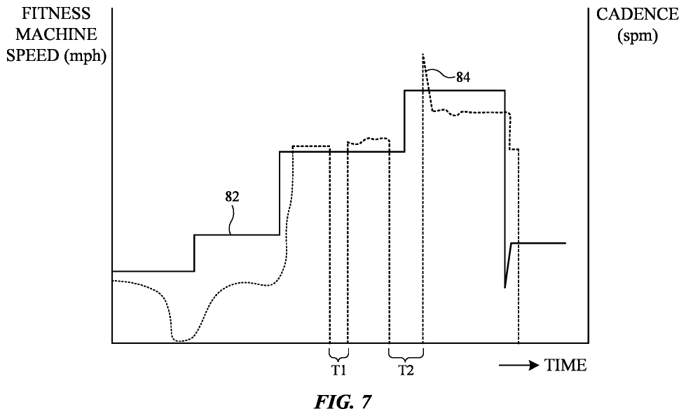

After the VO₂ max is calculated, the VO₂ max may be displayed on a display such as display 14, at optional step 80. In this way, the user may view their VO₂ max on the display. Alternatively or additionally, the VO₂ max value may be used to calculate other health metrics, may be communicated to third parties, such as healthcare professionals, or otherwise utilized. As previously described, because device 10 is receiving speed and/or grade information from external equipment 20, it may be necessary to ensure that the user of external equipment 20 is moving at the same speed as the external equipment setting. An example of this is shown in FIG. 7.

As shown in FIG. 7, a fitness/exercise machine, such as external equipment 20, may have a speed in miles-per-hour given by line 82. A user of external equipment 20 may have a cadence in steps-per-minute given by line 84. As shown, the user's cadence may generally coincide with the speed of the external equipment. However, at times T1 and T2, line 84 may be far below line 82, indicating that the user has stopped moving (i.e., the user's cadence is zero) or indicating that the user has stepped off of external equipment 20 and is moving slower than the reported speed. As such, the speed reported by external equipment 20 may be inaccurate, and should not be used in estimating the user's VO₂ max.

Any desired range/threshold may be used by a discordance detector, such as discordance detector 66, in determining whether a user's cadence is within an expected range. For example, the discordance detector may discard received speed and grade information if a user's cadence is zero steps-per-minute (spm). Alternatively, the discordance detector may discard the received speed and grade information if the user's cadence is less than 100 steps-per-minute. These values may reliably indicate that the user is not using the exercise equipment at the reported speed. Alternatively, however, the discordance detector may determine an expected cadence, such as 130 spm, 120 spm, 140 spm, or other value, based on the reported speed and grade. The discordance detector may then take corrective action (i.e., discarding or correcting the speed and grade information) if the cadence is outside of the expected cadence range by a predetermined amount, such as by 30 spm, 20 spm, 10 spm, or other desired amount.

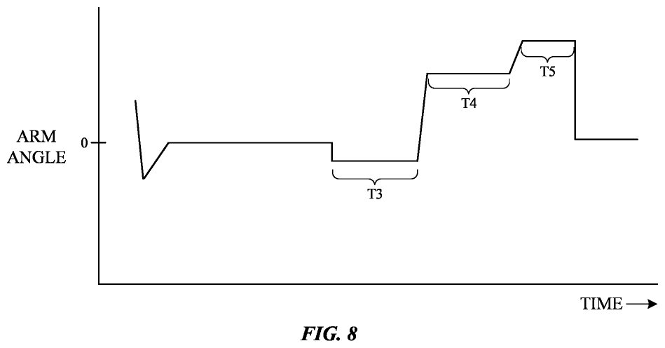

Although the discordance detector has been described as considering the user's cadence, the discordance detector may also consider other data to determine if the user is not moving at the same speed as exercise equipment. An example of an additional metric that a discordance detector, such as discordance detector 66, may consider is shown in FIG. 8.

As shown in FIG. 8, a user's arm angle may vary as a function of time. Sensors within a wearable electronic device, such as motion sensors (e.g., accelerometer 32 and/or gyroscope 34) and/or an orientation sensor, may track an angle of the user's arms as they are using exercise equipment. In some circumstances, a high or low arm angle that remains constant over a period of time may indicate that the user is holding onto side railings or handles at the front of the exercise equipment. As shown in FIG. 8, for example, the low arm angle at time T3 may indicate that a user is holding onto side rails of the equipment, while the high arm angle at times T4 and T5 may indicate that the user is holding onto handles at the front of the equipment. In either case, the user may be assisted by holding onto the rails, and using the speed of the exercise equipment may result in an inaccurate workrate calculation. Therefore, the speed and grade information may be discarded by the discordance detector, such as discordance detector 66, if it is determined that the user is holding onto a portion of the equipment. In this way, discordance detector 66 may ensure an accurate estimation of a user's VO₂ max as they are exercising indoors.

As described above, one aspect of the present technology is the gathering and use of information such as information from input-output devices. The present disclosure contemplates that in some instances, data may be gathered that includes personal information data that uniquely identifies or can be used to contact or locate a specific person. Such personal information data can include demographic data, location-based data, telephone numbers, email addresses, twitter ID's, home addresses, data or records relating to a user's health or level of fitness (e.g., vital signs measurements, medication information, exercise information), date of birth, username, password, biometric information, or any other identifying or personal information.

The present disclosure recognizes that the use of such personal information, in the present technology, can be used to the benefit of users. For example, the personal information data can be used to deliver targeted content that is of greater interest to the user. Accordingly, use of such personal information data enables users to calculated control of the delivered content. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the United States, collection of or access to certain health data may be governed by federal and/or state laws, such as the Health Insurance Portability and Accountability Act (HIPAA), whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, the present technology can be configured to allow users to select to “opt in” or “opt out” of participation in the collection of personal information data during registration for services or anytime thereafter. In another example, users can select not to provide certain types of user data. In yet another example, users can select to limit the length of time user-specific data is maintained. In addition to providing “opt in” and “opt out” options, the present disclosure contemplates providing notifications relating to the access or use of personal information. For instance, a user may be notified upon downloading an application (“app”) that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of information that may include personal information data to implement one or more various disclosed embodiments, the present disclosure also contemplates that the various embodiments can also be implemented without the need for accessing personal information data. That is, the various embodiments of the present technology are not rendered inoperable due to the lack of all or a portion of such personal information data.

The foregoing is illustrative and various modifications can be made to the described embodiments. The foregoing embodiments may be implemented individually or in any combination.

What is claimed is:

1. An electronic device configured to receive information from external equipment, comprising:
a housing;
a first sensor configured to measure a cadence;
a second sensor configured to measure a heart rate; and
a display in the housing, wherein the display is configured to display an estimated VO₂ max value based at least on the cadence, the heart rate, and the received information.

2. The electronic device defined in claim 1, wherein the received information comprises speed and grade information.

3. The electronic device defined in claim 2, wherein the electronic device further comprises:
communications circuitry configured to receive the speed and grade information from the external equipment.

4. The electronic device defined in claim 3, wherein the VO₂ max value is discarded if the cadence is outside of an expected cadence range.

5. The electronic device defined in claim 4, wherein the expected cadence range is determined based on the received speed and grade information.

6. The electronic device defined in claim 3, wherein the VO₂ max value is discarded if the cadence is less than 100 steps per minute.

7. The electronic device defined in claim 2, wherein the received information comprises image data from external camera equipment.

8. The electronic device defined in claim 7, wherein the image data is used to determine the speed and grade information.

9. The electronic device defined in claim 8, wherein the VO₂ max value is discarded if the cadence is outside of an expected cadence range and wherein the expected cadence range is determined based on the speed and grade information.

10. A method of estimating a VO₂ max value with an electronic device having a motion sensor and a heart rate sensor, the method comprising:
gathering cadence data using the motion sensor;
gathering heart rate data using the heart rate sensor;
receiving speed and grade information from external equipment; and
estimating the VO₂ max value based at least on the cadence data, the heart rate data, and the speed and grade information.

11. The method defined in claim 10, further comprising:
determining whether the cadence data is within a range of an expected cadence; and
discarding the VO₂ max value if the cadence data is not within the range of the expected cadence.

12. The method defined in claim 11, further comprising:
determining the expected cadence based on the speed and grade information from the external equipment.

13. The method defined in claim 12, wherein discarding the VO₂ max value if the cadence data is not within the range of the expected cadence comprises discarding the VO₂ max value if the cadence data is at least 30 steps-per-minute different from the expected cadence.

14. The method defined in claim 10, further comprising:
discarding the VO₂ max value if the cadence data is less than 100 steps-per-minute.

15. The method defined in claim 10, wherein receiving the speed and grade information from the external equipment comprises receiving the speed and grade information from a treadmill.

16. The method defined in claim 10, wherein receiving the speed and grade information from the external equipment comprises receiving the speed and grade information from a camera system.

17. The method defined in claim 10, further comprising:
measuring an arm angle using an orientation sensor; and
discarding the VO₂ max value if the arm angle indicates that the user is holding onto the external equipment.

18. An electronic device configured to receive speed and grade information from external equipment, the electronic device comprising:
a housing;
a first sensor in the housing that measures a cadence;
a second sensor in the housing that measures a heart rate; and
control circuitry configured to calculate a VO₂ max value based at least on the heart rate and the speed and grade information.

19. The electronic device defined in claim 18, further comprising:
a display configured to display the VO₂ max value.

20. The electronic device defined in claim 18, wherein the control circuitry is configured to discard the VO₂ max value in response to determining that the cadence is not within an expected cadence range.
